(19) United States
(12) Patent Application Publication
(10) Pub. No.: US 2006/0099720 A1
(43) Pub. Date: May 11, 2006

(54) Methods for performing differential capture proteomics

(76) Inventors: Paul Stroobant, Natick, MA (US); Robert McBurney, Natick, MA (US)

Correspondence Address: CLARK & ELBING LLP, 101 FEDERAL STREET, BOSTON, MA, 02110, US

(21) Appl. No.: 10/518,771

(22) PCT Filed: Jun. 20, 2003
(86) PCT No.: PCT/US03/19613

§ 371 Date: Oct. 27, 2005

Related U.S. Application Data
(60) Provisional application No. 60/390,655, filed on Jun. 20, 2002.

(51) Int. Cl. G01N 33/543

(52) U.S. Cl. 436/518

(57) ABSTRACT

Disclosed herein are improved methods for identifying, isolating, and comparing proteins and other biomolecules differing between two biological samples using affinity chromatography and phage display techniques.

### BACKGROUND OF THE INVENTION

The invention relates to methods for identifying and isolating proteins and other biological molecules using affinity chromatography and phage display techniques.

There is an increasing need, particularly in the fields of medicine and agriculture, to identify and characterize the molecules which participate in a wide variety of biological processes and to find new molecules capable of modulating these processes. One way to search for novel bioactive compounds is to screen libraries of natural materials or synthesized molecules, using assay techniques which can range in complexity from simple binding reactions to elaborate physiological preparations. Unfortunately, selecting molecules of interest from large ensembles or repertoires can be time-consuming and costly, and often only provides leads which require further investigation and development.

Recently, there have been several developments both in the generation of libraries and in the methods of their selection, which have improved the efficiency, and effectiveness of this approach. Phage display technology, largely developed in the 1990s, is an in vitro selection technique in which a protein (or peptide) is displayed on the surface of a phage virion, while the DNA encoding the protein is contained within the virion. This direct physical linkage between the displayed protein and the DNA encoding it allows for successive rounds of selection and amplification. Large phage display libraries (“PDLs”) can be generated and screened against target molecules. These PDLs may encompass an enormous number of different peptides, which represent potential ligands to a variety of macromolecules such as receptors, polypeptides, enzymes, carbohydrates, and antibodies. Individual phage can be captured from the libraries by virtue of the interaction of the displayed protein with a cognate ligand, and the captured phage can be amplified by infection of bacteria. Thus, phage display technology is a very powerful tool for the selection of peptides that bind to target molecules. These peptides may find numerous applications, for example, as antigens in vaccine compositions, as enzyme inhibitors, or as agonists or antagonists of receptors.

One strategy for developing diagnostic tests and drugs for treating a disease involves the identification of key cellular components, such as proteins, that are causally related to the disease process. This can often be accomplished by looking at differences in protein composition or protein activity between diseased and healthy individuals or between treated and untreated patients. Unfortunately, present methods of analyzing biomolecules are time-consuming and expensive, and suffer from inefficiencies in detection, imaging, purification, and analysis. Thus, there is a need for methods of detecting specific differences and changes between biological samples. Such methods would facilitate the identification of biological targets for diagnostic and drug development.

Although the genomics approach has advanced our understanding of the genetic basis of biological processes, it has significant limitations. For instance, the functions of products encoded by identified genes—and especially by partial cDNA sequences—are frequently unknown, and information about post-translational modifications of a protein can rarely be deduced from a knowledge of its gene sequence. It is now apparent that a large proportion of proteins undergo post-translational modifications (such as glycosylation and phosphorylation) that can profoundly influence their biochemical properties. Furthermore, protein expression is often subject to post-translational control, so that the cellular level of an mRNA does not necessarily correlate with the expression level of its gene product.

For these reasons, there is a need to supplement genomic data by studying the patterns of protein and carbohydrate expression, and of post-translational modification generally, in a biological or disease process through direct analysis of proteins, oligosaccharides, and other biomolecules. The burgeoning field of proteomics seeks to study variations in cellular protein levels between normal and diseased states by detecting and quantifying expression at the protein level, rather than the mRNA level. However, the proteomics approach faces numerous obstacles, including sample complexity, large relative abundance range, and quantification of proteins. Technical constraints have heretofore impeded the rapid, cost-effective, reproducible, and systematic analysis of proteins and other biomolecules present in biological samples.

### SUMMARY OF THE INVENTION

The present invention features improved methods for isolation and quantification of proteins and other biomolecules differing between samples over a wide range of relative abundance. These differences can include, for example, differences in the protein content of the two biological samples. Protein species that differ between two biological samples are referred to herein as “Difference Proteins.” The invention also provides for the identification of proteins as known species or as species with novel sequence or novel post-translational modifications and supplies a means for characterization and isolation of specific affinity reagents against such proteins.

In one aspect, the invention features improved methods for identifyig a biomolecule, e.g., a protein, using a technique known as Differential Capture Proteomics (“DCP”). The DCP method generally includes the steps of (a) adhering a first biological sample to a first support to create a first matrix including one or more biomolecules from the first sample; (b) adhering a second biological sample to a second support to create a second matrix including one or more biomolecules from the second sample; (c) exposing a library of binding species at least one time to the first matrix to create a first product including one or more binding species of the library, and (d) exposing the first product at least one time to the second matrix to create a second product, wherein a binding species present or absent in the second product is indicative of the abundance of the biomolecule in the first biological sample relative to the second biological sample.

The method may further include the steps of (e) exposing the library to the second matrix to create a third product including one or more binding species of the library; and (f) exposing the third product at least one time to the first matrix to create a fourth product, wherein a binding species present or absent in the fourth product is indicative of the abundance of the biomolecule in the second biological sample relative to the first biological sample. The second and fourth products may be compared to determine the abundance of the biomolecule in the first sample relative to the second sample.

The method may further include the steps of (g) combining the second and fourth products to produce a pooled product; (h) adhering at least a portion of the pooled product to a third support to provide a third matrix; (i) exposing the first biological sample at least one time to the third matrix to provide a fifth product; (j) exposing the second biological sample at least one time to the third matrix to provide a sixth product; and (k) comparing the fifth and sixth products to determine the abundance of the biomolecule in the first sample relative to the second sample.

Comparisons between products and identification of biomolecules or binding species may be achieved by an appropriate technique. Exemplary techniques include mass spectrometry (e.g., employing an ion trap detector), and nuclear magnetic resonance spectroscopy.

The various products formed by exposing a library or biological sample to a matrix may include either material which did not bind to the matrix (i.e., flow-through product) or material which bound to the matrix and was subsequently released (i.e., eluted bound product). The library or biological sample may be exposed to the matrix multiple times in order to produce the product.

The pooled product may be amplified prior to creation of the third matrix. Amplification procedures will depend on the nature of the binding species in the pooled product. Phage and nucleic acids are exemplary binding species that may be amplified using standard techniques.

The DCP methods allow for the identification of differences between two biological samples by using affinity chromatography and phage display techniques. The two biological samples may be from the same individual, different individuals, or even from two different types of organism. With these methods, a sample taken from a diseased organism can be compared against a sample taken from a non-diseased organism to identify differences in the expression of biomolecules within the two samples. Samples from organisms subjected to or not subjected to a chemical agent can likewise be compared. Exemplary chemical agents include pharmaceutical compounds, candidate pharmaceutical compounds, toxins, or any other chemical species. The identification of differences between such samples can lead to the identification of biological targets for therapy and to information useful for diagnostics and assessment of candidate therapeutic agents.

The biological samples to be compared can be taken from a wide variety of biological sources, including tissues, such as epithelial, connective, muscle, or nerve tissue, or cultured cell types derived therefrom. Alternatively, the biological samples may be taken from a bodily fluid, such as cerebrospinal fluid (CSF), blood, saliva, mucous, tears, pancreatic juice, seminal fluid, sweat, milk, bile, plasma, serum, lymph, urine, pleural effusions, bronchial lavage, ascites, or synovial fluid. In a particularly preferred embodiment, the bodily fluid is CSF.

In other embodiments, the biological samples are from an organ type, including skin, bone, cartilage, tendon, ligament, skeletal muscle, smooth muscle, heart, blood, blood vessel, brain, spinal cord, peripheral nerve, nose, trachea, lung, mouth, esophagus, stomach, intestine, kidney, uterus, ureters, urethra, bladder, hypothalamus, pituitary, thyroid, pancreas, adrenal gland, ovary, oviduct, vagina, mammary gland, testicle, seminal vesicle, penis, lymph, lymph node, lymph vessel, white blood cell, T-cell and B-cell.

Biomolecules assayed by the methods of the invention include proteins, nucleic acids, carbohydrates, fatty acids, lipids, steroids, prostaglandins, prostacyclins, or small organic molecules.

In a preferred embodiment of the invention, the library is a peptide-nucleic acid coupled library, such as a phage display library, most preferably an antibody library, or a recombinant display library, or synthetic peptide library. When a phage display library is employed in the DCP method, the process is referred to herein as Differential Phage Capture Proteomics or “DPCP.” When a phage display library is employed in the DCP process, the method preferably includes a step of amplification or expansion of the phage following binding selection. Selection for the most abundant phage species during the infection of host cells (e.g., E. coli.) and subsequent growth of the cells can be controlled by varying the relative concentrations of phage and bacteria/cells, the time for initial binding, the temperature, and the time for which the bacteria are grown.

In alternative embodiments, a library other than a peptide-nucleic acid coupled library may be used. In particular, the Differential Capture Analysis techniques described herein can be employed using oligonucleotide libraries, carbohydrate libraries, organic small molecule libraries, and other display libraries. When such libraries are employed, there is generally no direct amplification of the captured molecules. Instead, the species captured during the Differential Capture Process may be identified, for example, by mass spectrometry and/or nuclear magnetic resonance spectroscopy, and may then be synthesized according to the chemical techniques appropriate for the particular type of library utilized.

The matrices generated according to the present invention can be a phage or affinity capture device, for example an affinity column, such as a phage affinity column, or a column of immobilized proteins. Alternatively, the matrix can be in the form of a planar substrate, such as a biochip (e.g., a protein chip).

The present invention provides various improvements to the DCP methodology. In particular, the present invention features methods for increasing the efficiency of the detection of biomolecules, e.g., proteins. For example, the DCP process may include the step of derivatization of protein species present in a biological sample, thereby modifying functional groups on the biomolecules in order to facilitate immobilization onto a support, followed by exposing the immobilized biomolecules from the sample to a library, such as a phage display library. This derivatization step is controlled by varying, for example, the derivatizing agent, the concentrations of reactants, the temperature, and time for derivatization. The matrices of the invention can be prepared by covalently linking biomolecules in a biological sample to a support. The samples are preferably treated with chemical agents to denature the biomolecules, e.g., proteins, prior to being adhered to the support. The immobilization step, which may be performed either before or after phage binding, can also be controlled by adjusting the concentration of reactants, temperature, and time for immobilization. Capture events between biomolecules, e.g., proteins, and binding species, e.g., phage (when a phage display library is used in the DCP process), can likewise be controlled via manipulation of similar parameters.

The present invention also provides a method for improving the DPCP analysis by controlling the ratio of binding species to biomolecule in a sample being analyzed. In one embodiment, the method involves diluting the sample to a desired level prior to exposing the sample to the library. Preferably, the sample is serially diluted, and each of the serial dilutions, or a subset thereof, is exposed to a fixed concentration of binding species. In an alternative embodiment, the binding species-to-biomolecule ratio is controlled by exposing the sample to a series of progressively increasing concentrations of binding species.

By “adhering” is meant directly or indirectly linking a portion of one material to a portion of another material, either covalently or non-covalently.

By “amplifying” is meant increasing in number.

By “binding species” is meant any chemical or biological species that is capable of binding to another species. Exemplary binding species include phage, proteins (e.g., antibodies), and nucleic acids.

By “biological sample” is meant any solid or fluid sample obtained from, excreted by, or secreted by any living organism, including single-celled micro-organisms (such as bacteria and yeast) and multicellular organisms (such as plants and animals, for instance a vertebrate or a mammal, and in particular a healthy or apparently healthy human subject or a human patient affected by a condition or disease). A biological sample may be a biological fluid obtained from any site or substance of the organism (e.g., blood, plasma, serum, urine, bile, cerebrospinal fluid, aqueous or vitreous humor, or any bodily secretion), a transudate, an exudate (e.g., fluid obtained from an abscess or any other site of infection or inflammation), or fluid obtained from a joint (e.g., a normal joint or a joint affected by disease such as rheumatoid arthritis, osteoarthritis, gout or septic arthritis). Alternatively, a biological sample can be obtained from any organ or tissue (including a biopsy or autopsy specimen) or may comprise cells (whether primary cells or cultured cells) or medium conditioned by any cell, tissue or organ. If desired, the biological sample may be subjected to preliminary processing, including but not limited to preliminary separation techniques and/or denaturation. For example, cells or tissues can be extracted and subjected to subcellular fractionation for separate analysis of biomolecules in distinct subcellular fractions, e.g., proteins or drugs found in different parts of the cell. See Deutscher (ed.), Methods In Enzymology 182:147-238 (1990). Similarly, immunoprecipitation can be performed to identify antigenically related biomolecules such as proteins.

By “exposing” is meant allowing contact to occur between two materials.

By “individual” or “subject” is meant a single-celled or multicellular organism, such as a plant, animal, fungus, protozoan, or bacterium. In a preferred embodiment, the individual is a mammal, most preferably a human or other primate species.

By “library” is meant a diverse population of molecules. In preferred embodiments, the library has at least 10⁵, preferably at least 10⁸, more preferably at least 10¹⁰, and most preferably at least 10¹² different molecular species having, for example, a nucleic acid and/or an amino acid component.

By “matrix” is meant a plurality of polymer sequences (e.g., proteins, oligonucleotides, and polynucleotides) or other biomolecules which are associated with the surface of a support. Examples of matrices include a protein affinity column and a phage affinity column.

By “peptide-nucleic acid coupled library” is meant a collection of peptides wherein each peptide is linked (directly or indirectly) to the DNA that encodes the peptide. An example of a peptide-nucleic acid coupled library would be a phage display library (“PDL”).

The terms “peptide,” “protein,” and “polypeptide” are used interchangeably herein and refer to any chain of two or more amino acids joined to each other by peptide bonds or modified peptide bonds, regardless of post-translational modification (e.g., glycosylation or phosphorylation).

The present invention is useful for identifying and analyzing proteins, but is more generally applicable to the identification and analysis of any biomolecule. As used herein, the term “biomolecule” refers to any organic molecule that is present in a biological sample, and includes peptides, polypeptides, proteins, fatty acids, oligosaccharides, lipids, steroids, prostaglandins, prostacyclins, and nucleic acids (including DNA and RNA).

By “substrate” or “support” is meant any porous or non-porous water insoluble material, which is preferably rigid or semi-rigid. The surface can have any one of a number of shapes, such as membranes, filters, chips, slides, wafers, fibers, magnetic or nonmagnetic beads, gels, tubing, strips, plates, rods, polymers, particles, microparticles, capillaries, and the like. The substrate can have a variety of surface forms, such as wells, trenches, pins, channels and pores, to which polypeptides, polynucleotides, or other biomolecules are bound. The substrate can be hydrophilic or capable of being rendered hydrophilic and includes inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), etc.; either used by themselves or in conjunction with other materials; glass available as Bioglass, ceramics, metals, and the like. Natural or synthetic assemblies such as liposomes, phospholipid vesicles, and cells can also be employed. A commonly used support is Controlled Pore Glass (CPG), which consists of a glass matrix prepared uniformly with pores of defined size. Immobilization of proteins and other biomolecules on a substrate or surface may be accomplished by well-known techniques, commonly available in the literature.

Other features and advantages will be apparent from the following description and the claims.

### BRIEF DESCRIPTION OF THE DRAWINGS

FIG. 1 is a schematic diagram showing the preparation of protein affinity matrices from two different biological samples.

FIG. 2 is a schematic diagram showing an initial capture of phage by the protein affinity matrices.

FIG. 3 is a schematic diagram showing a subsequent capture step, in which phage that were captured by the first affinity column are then run through the second column, and vice versa.

FIG. 4 is a schematic diagram showing the preparation of a set of phage affinity matrices by pooling and amplifying the flow-through phage.

FIG. 5 is a schematic diagram showing a third capture step in which biological samples from two different individuals are passed through a set of phage affinity matrices/columns.

FIG. 6 is a schematic diagram showing the identification of proteins that differ between two separate biological samples by eluting proteins that bound to phage affinity columns and analyzing the proteins via mass spectrometry.

FIG. 7 is a schematic diagram showing the isolation of affinity reagents against proteins that differ between two biological samples.

FIG. 8 is a schematic diagram showing a cycle for depletion of the most abundant common proteins within two different biological samples.

FIG. 9 is a schematic diagram showing the capture of difference proteins following the depletion of most abundant common proteins from a pair of biological samples.

FIG. 10 is a schematic diagram summarizing the process of differential phage capture proteomics as illustrated in FIGS. 1-9.

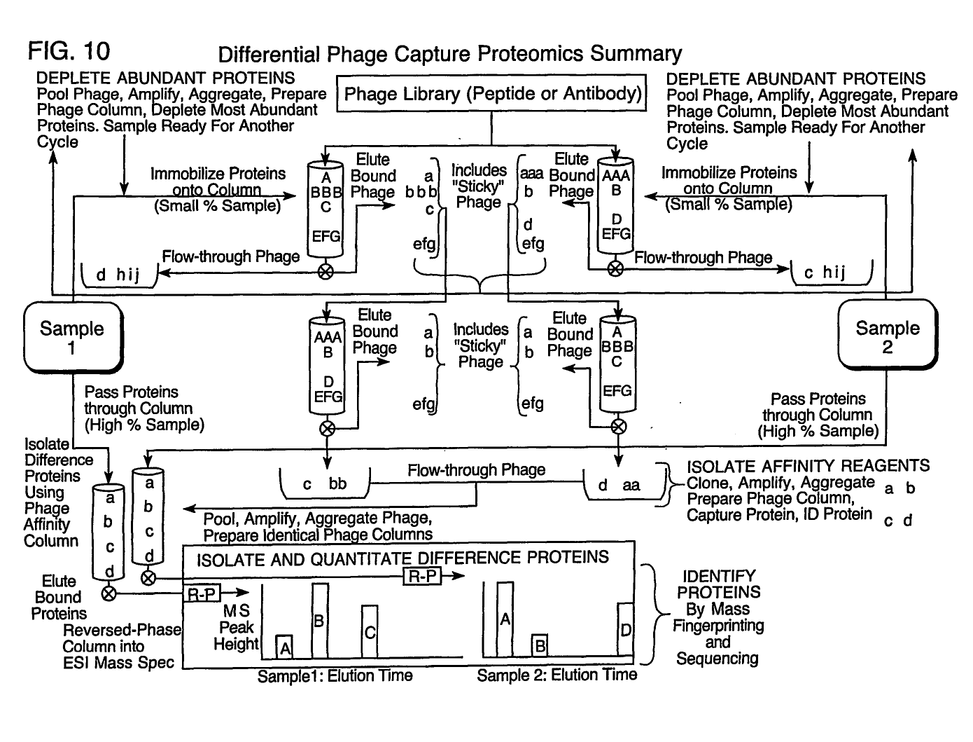

FIG. 11 is a schematic diagram showing the limited capability of 2D gels for the differential analysis of human plasma.

FIG. 12 is a schematic diagram illustrating how DPCP analysis can be optimized though adjustment of the phage-to-protein ratio.

FIG. 13 is a chart mathematically showing the isolation of phage against species changing between two samples.

FIG. 14 is a schematic diagram showing how a range of serial dilutions of samples with fixed phage concentration allows for the analysis of proteins within a desired range of relative abundance.

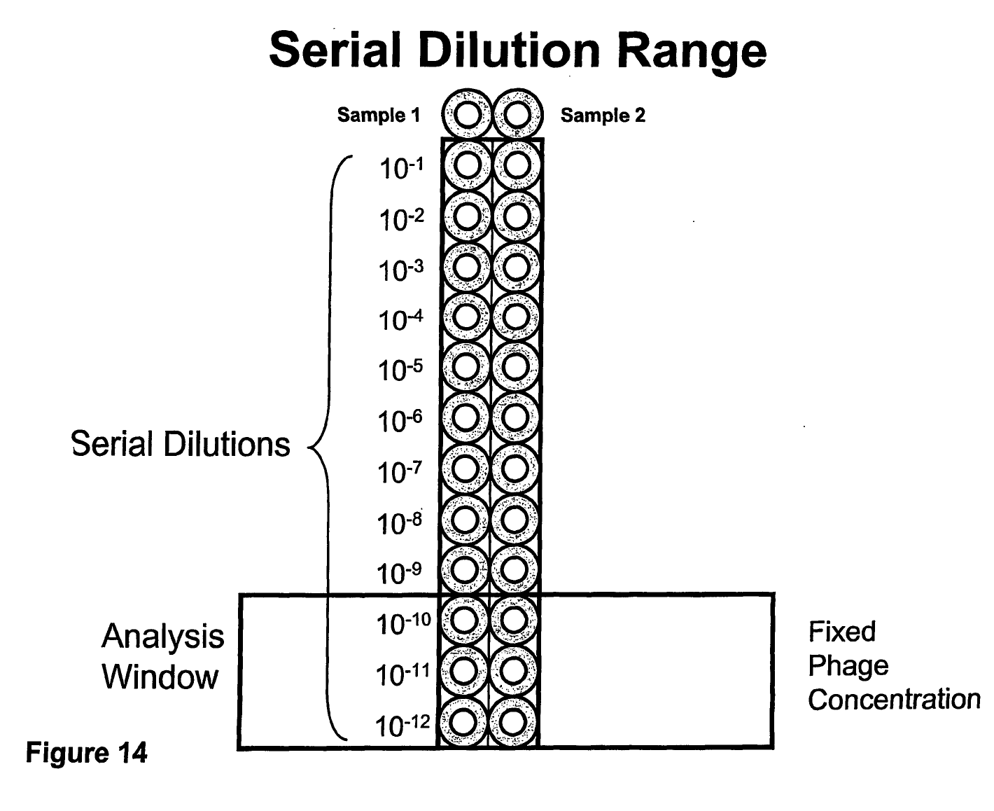

### DETAILED DESCRIPTION OF THE INVENTION

In developing new methods for diagnosing and treating a disease, it is important to identify the key cellular components, such as proteins and other biomolecules, that are associated with the disease. One way to identify such components is to look for differences in protein expression between diseased and healthy individuals.

The present invention provides improved methods for comparing two biological samples in order to determine the differences in proteins and other biomolecules that are present in the samples. In a preferred embodiment, a pair of protein affinity matrices are prepared from the two biological samples to be compared. A phage display library is exposed to the matrices in a series of capture steps which results in the isolation of phage that bind to those proteins that are different between the two samples (i.e., the “difference proteins”). These phage are amplified and used to prepare a set of phage affinity matrices. The biological samples being compared are then exposed to these phage matrices in order to capture differentially expressed proteins. By comparing a sample taken from a diseased subject to a sample taken from a healthy subject, one can identify biological targets of therapeutic and diagnostic importance and lead structures for drug development. For example, a receptor that is found to exist on only a sample taken from a diseased individual, or at a different concentration in the diseased sample, may serve as a potential target for diagnosis or treatment of the disease. In addition, a ligand found to have high affinity and specificity for the receptor provides a lead structure for drug development. Furthermore, a protein species which changes its distribution, level, or characteristics during treatment may provide an indication of beneficial or toxic effect in an animal, such as a human patient.

Samples can be taken from a wide variety of organs types, including but not limited to skin, bone, cartilage, tendon, ligament, skeletal muscle, smooth muscle, heart, blood, blood vessel, brain, spinal cord, peripheral nerve, nose, trachea, lung, mouth, esophagus, stomach, intestine, kidney, uterus, ureters, urethra, bladder, hypothalamus, pituitary, thyroid, pancreas, adrenal gland, ovary, oviduct, vagina, mammary gland, testicle, seminal vesicle, penis, lymph, lymph node, lymph vessel, white blood cell, T-cell and B-cell. Other suitable sample sources include, but are not limited to, epithelial, connective, muscle, or nerve tissue, or bodily fluids, such as cerebrospinal fluid (CSF), blood, saliva, tears, mucous, pancreatic juice, seminal fluid, sweat, milk, bile, plasma, serum, lymph, urine, pleural effusions, bronchial lavage, ascities, or synovial fluid.

The source of the sample is chosen based on a variety of factors, such as the nature of the disease or condition being studied. For instance, CSF may be taken to study a disease of the central nervous system, while pancreatic juice may be taken to study a disease of the pancreas. In a disease state such as cancer, any or all of the types of tissues or cells which are related directly to the particular type of cancer (e.g., lymph for lymphoma, etc.) may be analyzed. Methods for properly collecting and storing various biological samples are known in the art, and may vary depending on the nature of the sample.

Preparation of Affinity Matrices Using Biological Samples

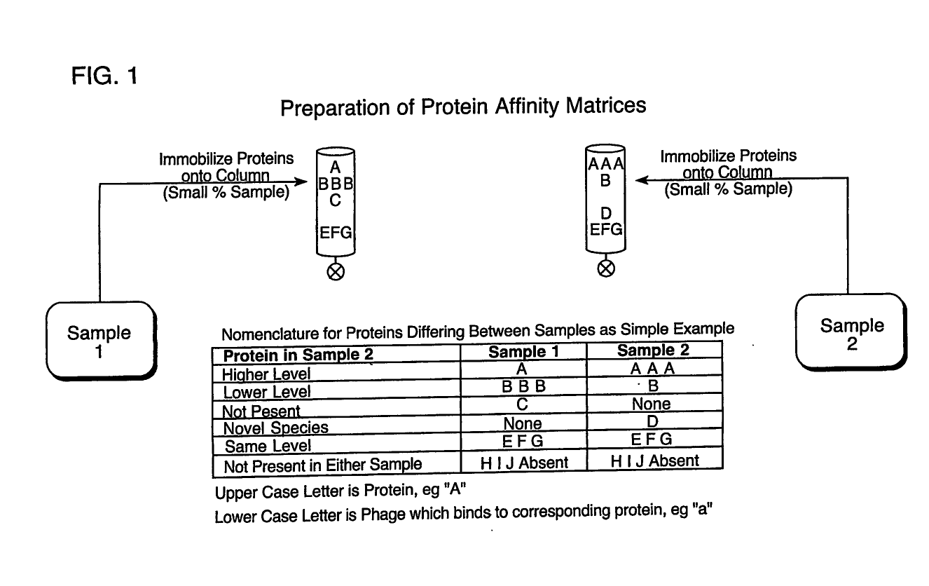

After the biological samples have been collected, a set of matrices are created by adhering each of the samples to a separate support (FIG. 1). In one embodiment of the invention, the matrix is an affinity matrix that includes a solid support or gel to which is attached a multiplicity of different proteins or other biomolecules. Suitable support materials include, but are not limited to paper, glasses, ceramics, metals, metalloids, polyacryloylmorpholide, various plastics and plastic copolymers such as NYLON™, TEFLON™, polyethylene, polypropylene, poly(4-methylbutene), polystyrene, polystyrene, polystyrene/latex, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and controlled-pore glass (Controlled Pore Glass, Inc., Fairfield, N.J.), aerogels (see, e.g., Ruben et al., J. Materials Science 27:4341-4349 (1992); Rao et al., J. Material. Science 28:3021 (1993); Back et al., J. Phys. D. Appl. Phys. 22:7309-734 (1989); Kim & Jang, J. Am. Ceram. Soc. 74:1987-92 (1991) and the like, and other materials generally known to be suitable for use in affinity columns. In a preferred embodiment, the support is a streptavidin sepharose column. However, screening can be carried out on other solid phases or in solution.

Biomolecules, such as proteins, can easily be attached to a solid substrate where they act as immobilized ligands that interact with complementary molecules present in a solution contacted to the substrate. The source to be screened, for example a phage display library, is passed over the affinity matrix, allowing target molecules to be captured by the immobilized ligands. Unbound components can be washed away from the bound complex to either provide a solution lacking the target molecules bound to the affinity column, or to provide the isolated target molecules themselves. After unbound background substances are washed away, the bound material is eluted, often in an eluent that weakens the association between the target and the ligand. The biomolecules captured in a affinity matrix can be separated and released by denaturation either through heat, adjustment of salt concentration, or the use of a destabilizing agent such as formamide, TWEEN™-20 denaturing agent, or sodium dodecyl sulfate (SDS).

Many techniques are known in the art for attaching proteins and other biomolecules to a substrate or support, such as those described in, for example, Bioconjugation: Protein Coupling Techniques for the Biomedical Sciences, M. Aslam, A. Dent Groves Dictionaries, Inc. New York, N.Y. (1998). Any suitable derivatization or solid phase binding method may be used. In a preferred embodiment, the proteins of the biological sample are adhered by biotinylating the proteins and using a substrate or support that includes avidin or an avidin-related compound (e.g., streptavidin). The biotin specifically binds to the avidin-related compound thereby attaching the proteins to the substrate. Other well known specific binding pairs may also be used as a means for attaching the sample proteins to the support.

Prior to adhering the samples to the support, the proteins of the sample may be denatured to allow for a more complete analysis. A variety of dissociative methods are known in the art and may be used to break up protein complexes, solubilize proteins, and unfold proteins within the samples. These methods include, for example, treatment with guanidine HCL, formic acid, various chaotropes, detergents, heating, phase partitioning, and derivatization. Alternatively, if the associations of proteins in the sample are to be preserved, such treatments are omitted and the protein complexes may optionally be crosslinked to increase stability. By preserving associations, phage against one member of a protein complex may allow for the isolation and identification of more than one component of a complex.

Peptide-Nucleic Acid Coupled Libraries

In accordance with the present invention, the protein affinity matrices described above are used to screen, for example, a peptide-nucleic acid coupled library, which is made up of a collection of peptides, wherein each peptide is linked to the DNA encoding it. In a preferred embodiment, the library is a phage display library. Display technology represents a collection of methods for creating libraries of modularly coded biomolecules that can be screened for desired properties. Two of the most important characteristics of display technologies are extremely high detection sensitivity and the ability to determine the structure of a desired compound rapidly after initial screening. A variety of phage libraries can be used, in the present invention, including immune or nonimmune, and single-chain Fv or Fab fragment antibody libraries; and recombinant-display or synthetic peptide libraries. Examples of suitable phage display libraries and techniques for their preparation are well known in the art and described in, for example, Barbas, F., et al., Phage Display: A Laboratory Manual, Cold Spring Harbor, N.Y. (2001). Other libraries which can be used are described by Li, M., “Applications of display technology in protein analysis,” Nature Biotechnology 18:1251-1258 (2000).

Peptide libraries comprise vast numbers of peptides of a given length, whose sequences have been randomly generated to vary the amino acid residues at each position. The usual goal for using such libraries is to select high affinity binders from the typical pool of binders that is found against nearly all proteins that are screened. In its simplest form, the method of the invention uses a single binder of moderate/low affinity, which is selected during the process for optimal protein capture and release

Phage display libraries can be selected based on their particular properties, depending on the type of analysis required and the properties of the affinity reagents to be isolated. For example, the choice between peptide and antibody phage display libraries is related to whether a desired affinity reagent is a peptide or an antibody. The library used preferably contains as large and diverse a population of binders specific for the chosen sample as possible. Compatible mixtures of libraries can be used to capture as many species as possible from the chosen sample. Ultimately, “panproteomic” and “proteomic subset” libraries can be developed which contain binders for all known species in any particular type of sample.

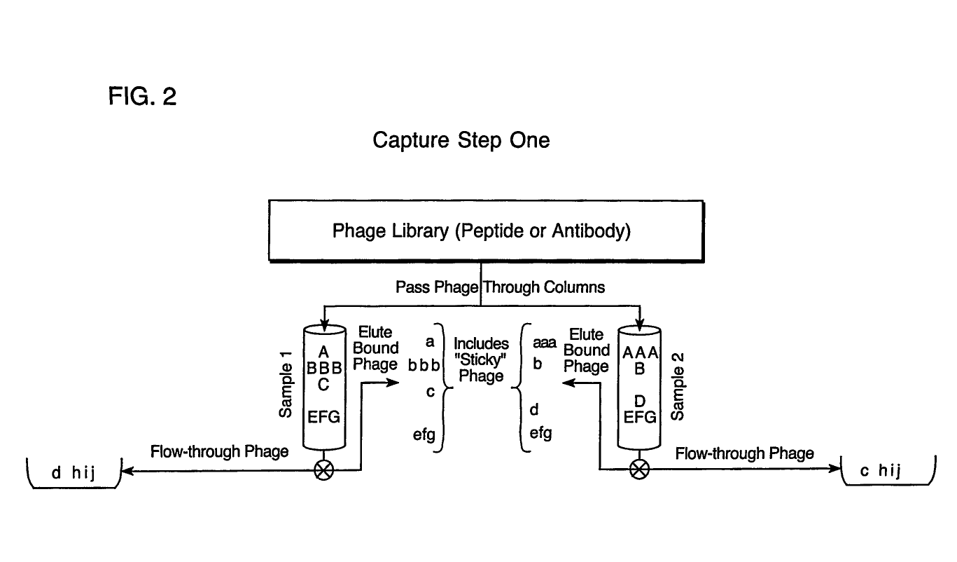

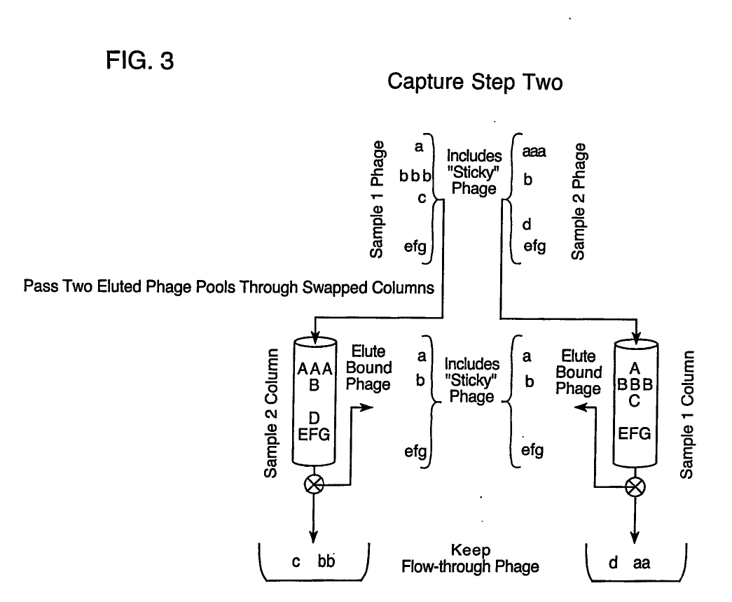

The phage library is passed through the protein affinity matrices generated from two different biological samples in order to capture phage that bind the proteins that differ between the two samples. The sequence of phage exposure is shown in FIGS. 2 and 3. First, the phage library is exposed to a protein affinity matrix prepared from the first biological sample. The unbound phage is washed away, and the bound phage is released and then exposed to a protein affinity matrix prepared from the second biological sample. The flow-through from this second exposure step is retained and contains phage that bind to proteins present in the first biological sample but not the second sample.

An identical phage library is exposed to the affinity matrices in the reverse order, i.e., the library is first exposed to a protein affinity matrix prepared from the second sample. The unbound phage is washed away and the bound phage is eluted and then exposed to a protein affinity matrix prepared from the first sample. The flow-through from the second exposure step is retained and contains phage that bind to proteins present in the second sample, but not the first sample.

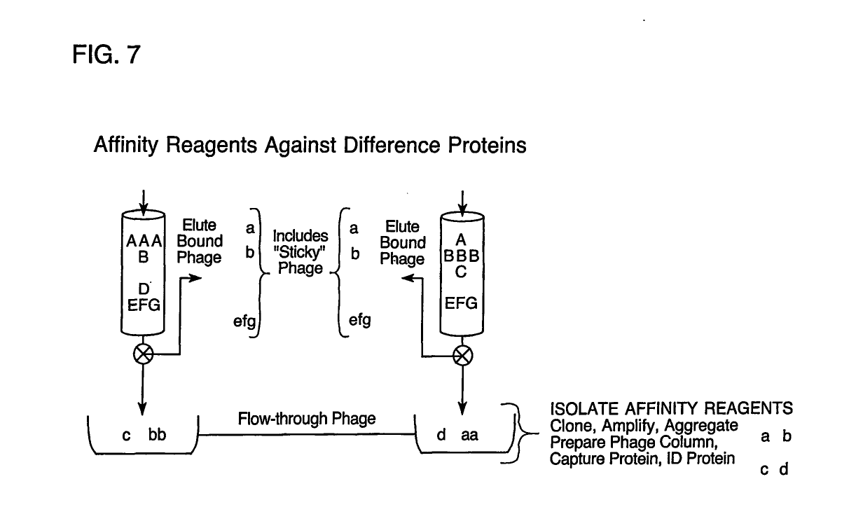

Thus, the phage library goes through two capture steps. In the first step, the phage library is passed through the protein affinity matrices prepared from the biological samples, and the bound phage are recovered. In the second capture step, the phage, which bound to the protein affinity matrix prepared from the first sample, are exposed to the protein affinity matrix prepared from the second sample. Conversely, phage, which bound to the protein affinity matrix prepared from the second sample, are exposed to the protein affinity matrix prepared from the first sample. This combination of capture steps results in the isolation of phage that are capable of binding to those proteins which are different between the first and second samples. The flow-through phage from the second capture step may be pooled, amplified, and used to prepare a set of identical phage affinity matrices for further screening. In addition, the phage may be used to isolate affinity reagents against the “difference proteins” (See FIG. 7).

Phage Affinity Matrices

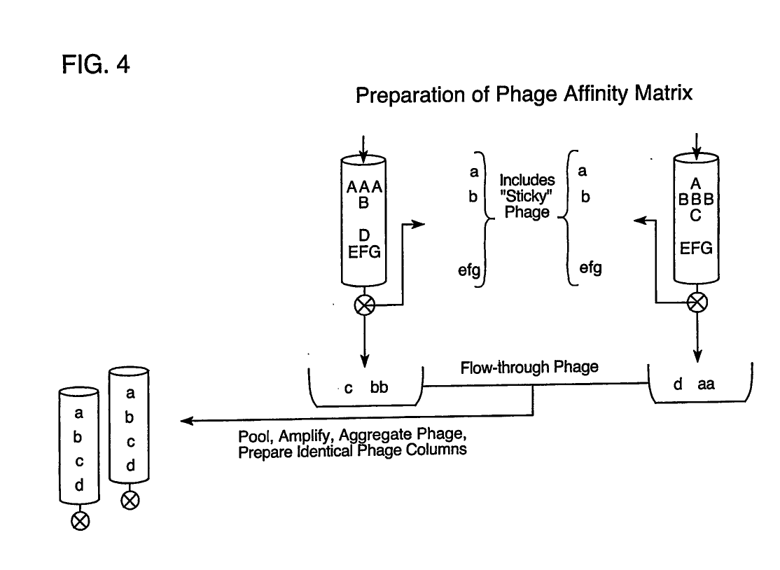

As discussed above, the phage which have been selected from the phage display library by exposure to protein affinity matrices are used to generate a set of phage affinity matrices (FIG. 4). Methods for preparing phage as an affinity matrix are known in the art and described, for example, by Smith et al., Journal of Immunological Methods 215:151-161 (1998). These phage affinity matrices can be used to directly screen the biological samples being compared. Prior to passing the samples through the phage matrix, they may be treated, as discussed above, using any of a variety of dissociative methods to break up protein complexes, solubilize proteins, and unfold proteins to allow a more complete analysis. Cross-linked filamentous phage can be successfully employed directly for affinity purifications, and the direct use of aggregated phage encoding an affinity capture peptide avoids the need to decode the appropriate peptide sequence, synthesize it and then prepare an affinity capture matrix, although this can be done if desired.

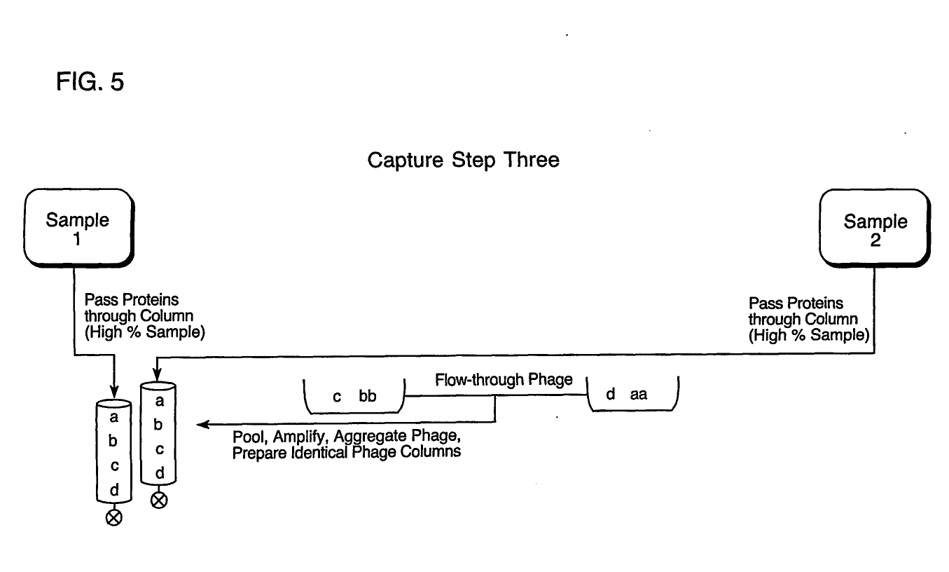

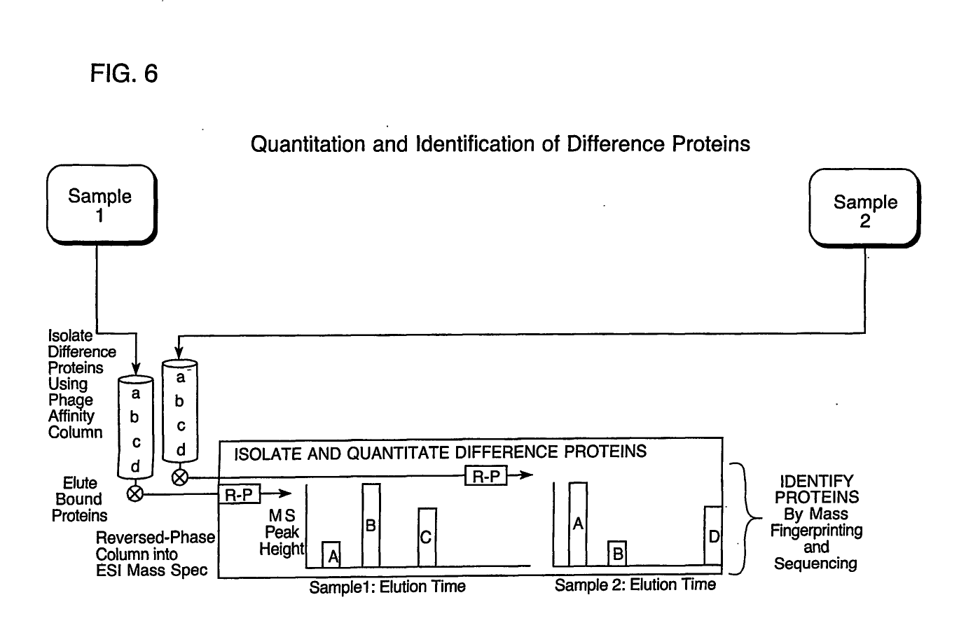

As shown in FIG. 5, biological samples from two different individuals are exposed to the phage affinity matrices. The proteins that bind to the phage matrices include those proteins which are different between the two samples. Proteins which fail to bind to the phage matrices are washed away, and the bound proteins are eluted and analyzed (FIG. 6), using any of a variety of identification and quantification methods known in the art. (See Gygi, S. P., et al., Nature Biotechnology 17:994-999 (1999); Gygi, S. P., et al., Curr. Opin. Biotechnol. 11:396-401 (2000); Oda, Y, et al, Proc. Natl. Acad. Sci. USA 96:6591-6596 (1999); Mirgorodskaya, O. A., et al., Rapid Commun. Mass Spectrom. 14:1226-1232 (2000); Munchbach, M., et al, Anal. Chem. 72:4047-4057 (2000); Link, A. J. et al., Electrophoresis 18:1314-1334 (1997); Washburn et al., Nature Biotechnology 19: 242-247 (2001)). In a preferred embodiment, the eluted proteins are passed through a reverse phase column into an ESI mass spectrometer. The proteins are identified by mass fingerprinting and sequencing. In addition to identifying proteins that are present in one sample but not the other, one skilled in the art will appreciate that the method of the invention can also be used to determine differing levels of a protein between two samples.

Depletion of Most Abundant Common Proteins

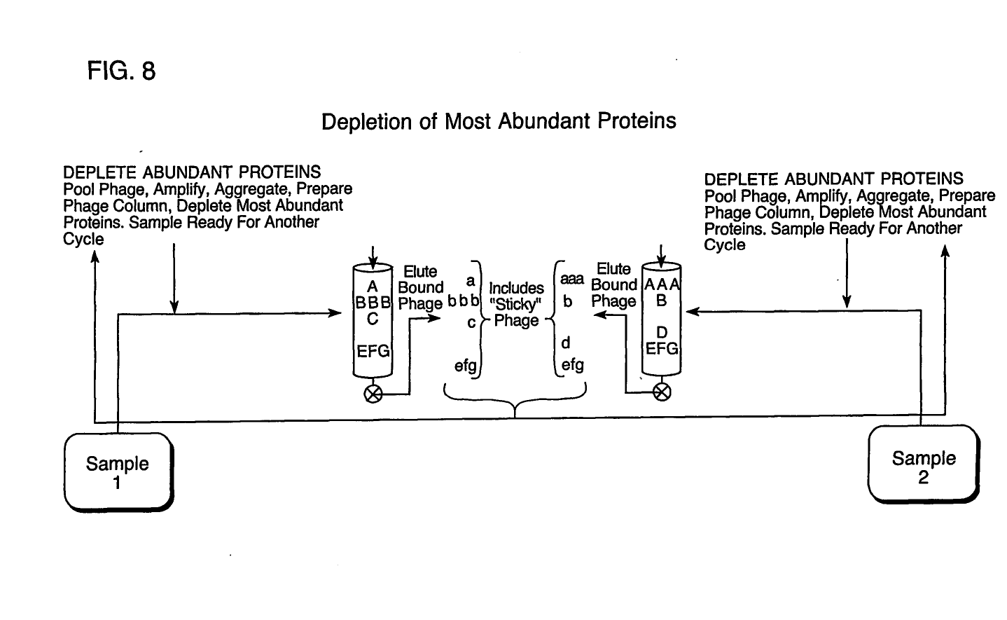

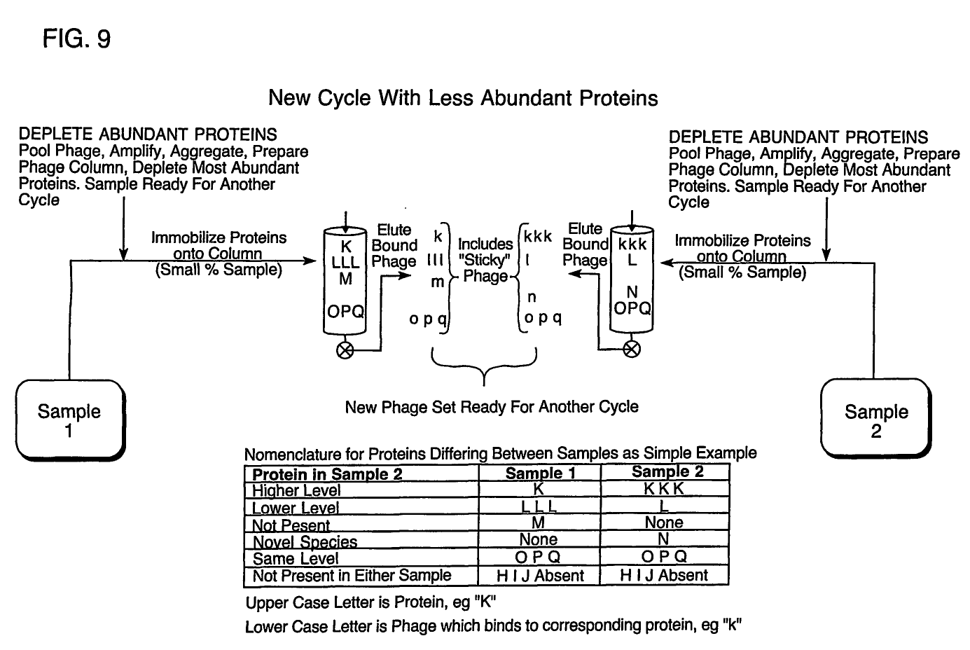

As shown in FIG. 8, in one embodiment of the invention, the most abundant common proteins contained within the biological samples can be depleted by recovering the bound phage after the initial capture step (FIG. 2), amplifying the phage, preparing phage affinity columns, and repeating the cycle using the methods described above and in the Example below. The steps shown in FIGS. 8 and 9 can be continuously repeated until the limits of detection of the analytical device have been reached. Once the most abundant common proteins have been depleted, the differences in the biological samples can be analyzed according to the techniques described herein, using samples containing less abundant common proteins. This allows for identification of proteins which may be present in very low quantities in the biological samples.

Gain Control for Fold Difference Between Protein Species

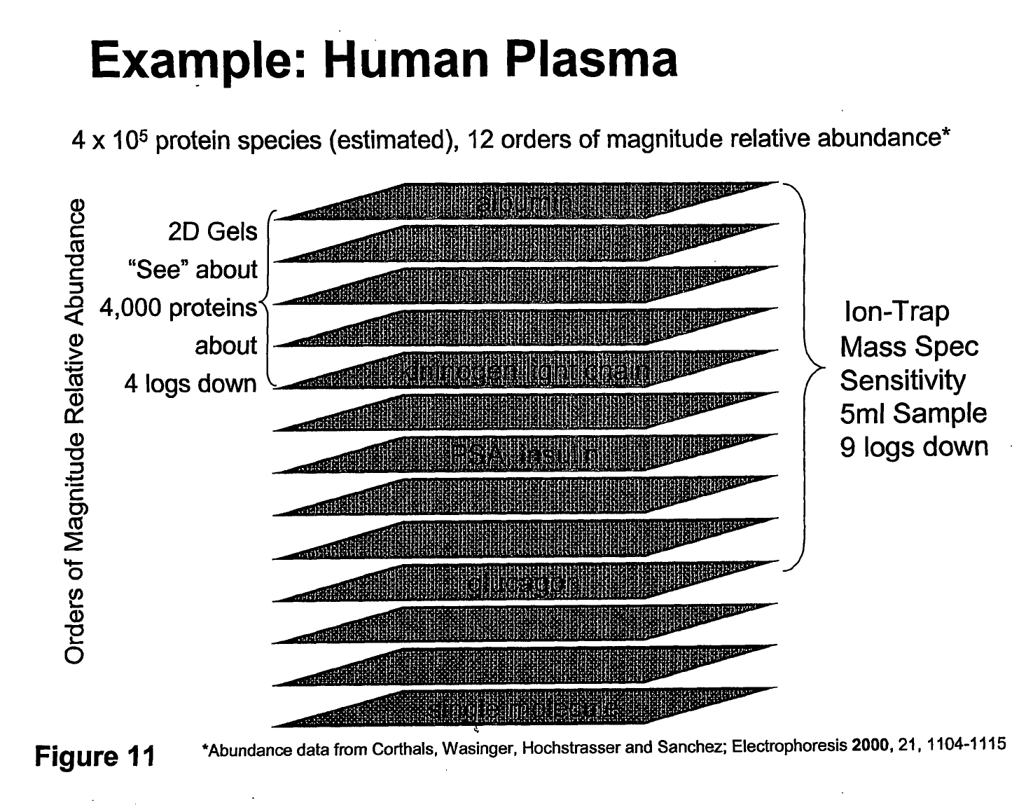

Methods for the analysis of the differences in protein species between any two biological samples as currently practiced using 2D gels and mass spectrometry are able to identify only a small proportion of the difference species present in most samples. Post-detection studies of the numbers and relative abundances of the difference species have been straightforward, since these methods typically detect fewer than 50 differences between fewer than 4,000 species observed as stained spots on the gels. In contrast, samples such as serum may contain 400,000 or more species with hundreds to thousands of differences covering a relative abundance range of 12 orders of magnitude (FIG. 11). It is notable that an ion-trap mass spectrometer is able to detect up to five orders of magnitude below what can be detected with 2D gels, and therefore enables DPCP to reach unprecedented levels of sensitivity for the differential analysis of protein samples.

DPCP, for the first time, offers a much more complete view of the differences in protein species between any two samples with the capability of identifying hundreds to thousands of difference species covering a wide range of concentrations and fold-differences. In addition, affinity reagents and purified protein reagents for the difference species are produced as an integral part of the process. For some studies, it will be important to establish the identities, concentrations, fold differences and reagents for all of the detectable difference species, but for others it will be essential to simplify the analysis to identify only the larger-fold changes for particular abundance ranges. Since DPCP is an iterative process, in which successive cycles of analysis focus on particular abundance ranges of difference protein species, there are opportunities to control the efficiency of detection for each abundance range. The following are examples of points at which the relative efficiency of the process can be controlled:

1. The Initial Derivatization of Proteins

The first step in the DPCP method, as shown in FIG. 1, involves the preparation of protein affinity matrices. This step can begin with the derivatization of the protein species present in the sample. In this derivatization step, chemical modifications are made to specific functional groups on the polypeptide chains to immobilize them in order to bind specific phage particles from a phage display library. The efficiency of the derivatization step can be controlled by varying the nature of the derivatizing agent, the concentrations of the reactants, and the temperature, and the time for derivatization (Bioconjugation: Protein Coupling Techniques for the Biomedical Sciences, M. Aslam, A. Dent Groves Dictionaries, Inc. New York, N.Y. (1988)).

2. The Immobilization of Proteins

The next step involves immobilization of the protein species from the biological sample onto a support (e.g., column) to enable phage binding to occur, or to capture the proteins following phage binding. The efficiency of this immobilization step can be controlled by varying the concentration of the reactants, the temperature and the time for immobilization, and performing the immobilization before or after phage binding (Bioconjugation: Protein Coupling Techniques for the Biomedical Sciences, M. Aslam, A. Dent Groves Dictionaries, Inc. New York, N.Y. (1988)).

3. The Capture Events between Proteins and Phage

After immobilization of the protein species, the subsequent steps in the DPCP process, shown in FIGS. 2-6, involve protein species capturing phage, and phage species capturing proteins. The efficiency of these processes can be controlled by varying the concentrations of the reactants, the temperature, and the time for binding (Bioconjugation: Protein Coupling Techniques for the Biomedical Sciences, M. Aslam, A. Dent Groves Dictionaries, Inc. New York, N.Y. (1988)).

4. The Expansion of Phage Following Binding Selection

Selection for the most abundant phage species during the infection and subsequent growth of E. coli can be controlled by varying the relative concentrations of phage and bacteria, the time for initial binding, and the temperature, and the time for which the bacteria are grown (Phage Display: A Laboratory Manual, Cold Spring Harbor, N.Y. (2001); Antibody Phage Display: Methods and Protocols, R. M. O'Brien and R. Aitken, Humana Press, Totowa, N.J. (2002)).

5. The Manipulation of Data in the Mass Spectrometer

A considerable degree of control is possible over the concentrations and the fold-differences of the protein species that emerge from the DPCP process at the level of analysis by mass spectrometer. The degree of control is particularly powerful using an ion-trap instrument, with which it is also possible to partially select the abundance ranges using exclusion tables (Proteome Research: Mass Spectrometry, P. James, Springer-Verlag, New York, N.Y. (2001)).

Protein Abundance Window Analysis

The DPCP methodology utilizes a column swapping procedure (See Capture Step Two, shown in FIG. 3) which requires that the phage that bind to difference proteins changing in concentration between any two samples are initially present in excess over the numbers of difference proteins. Difference proteins where this requirement is not met are “invisible” to the process, i.e., are not detected. For some samples, a large number of phage may be required to saturate high abundance proteins. In addition, due to the extreme sensitivity of the DPCP process to any changes in protein species composition, a small change in the concentration of a very abundant protein, which would generate a large number of phage after the column subtraction process (Capture Step Two, see FIG. 3), may swamp a large change in a much less abundant protein, since the larger number of phage may dominate the amplification step, thereby making it more difficult to detect even very large differences in the concentration of less abundant proteins. In order to address these situations, the present invention provides a novel method referred to as “Protein Abundance Window Analysis.”

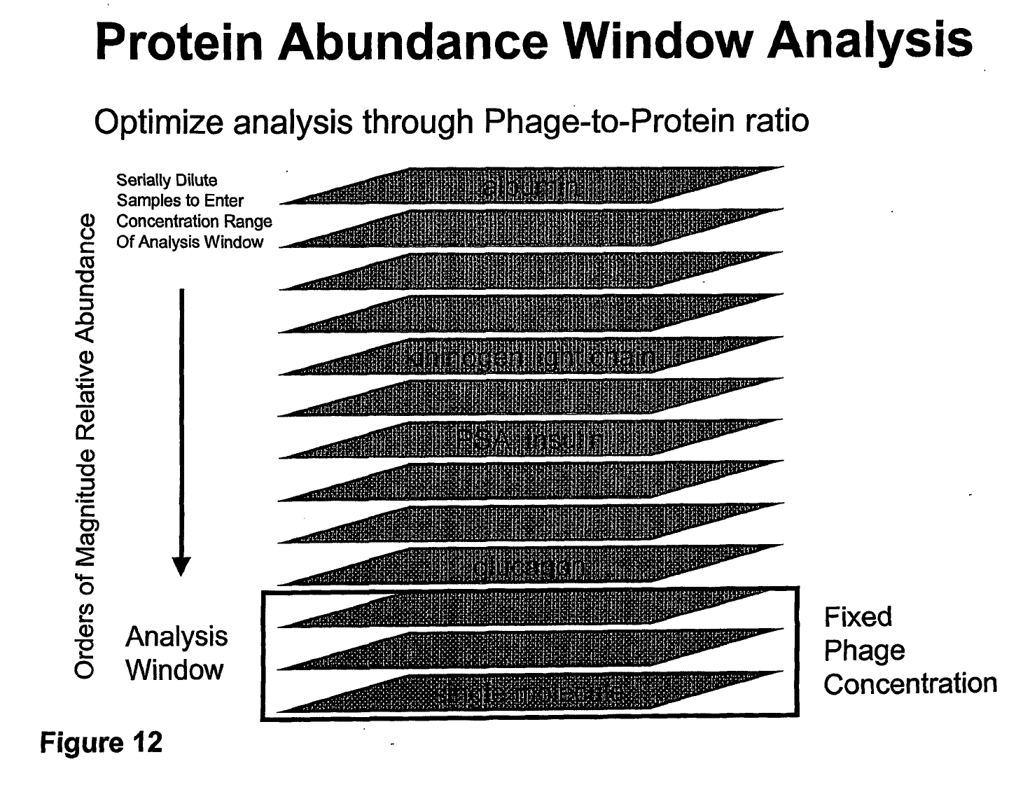

The general goal of this method is to optimize the DPCP analysis by manipulating the ratio of phage to proteins in the samples (FIG. 12). The phage-to-protein ratio is adjusted in order to achieve a condition where the phage particles are present in excess of the sample protein being analyzed. For samples containing numerous proteins over a wide range of relative abundances, adjustment of the phage-to-protein ratio is preferably accomplished by serially diluting the sample prior to exposing it to the phage. As the sample becomes progressively more dilute, the levels of the more abundant proteins in the sample decrease relative to a fixed concentration of phage. Difference Proteins which, at lesser dilutions, are present in excess of the number of phage, will, at higher dilutions, be present at levels less than the number of phage. Once the more abundant proteins reach the point where they are present in an amount less than number of phage, they become amenable to Differential Capture analysis.

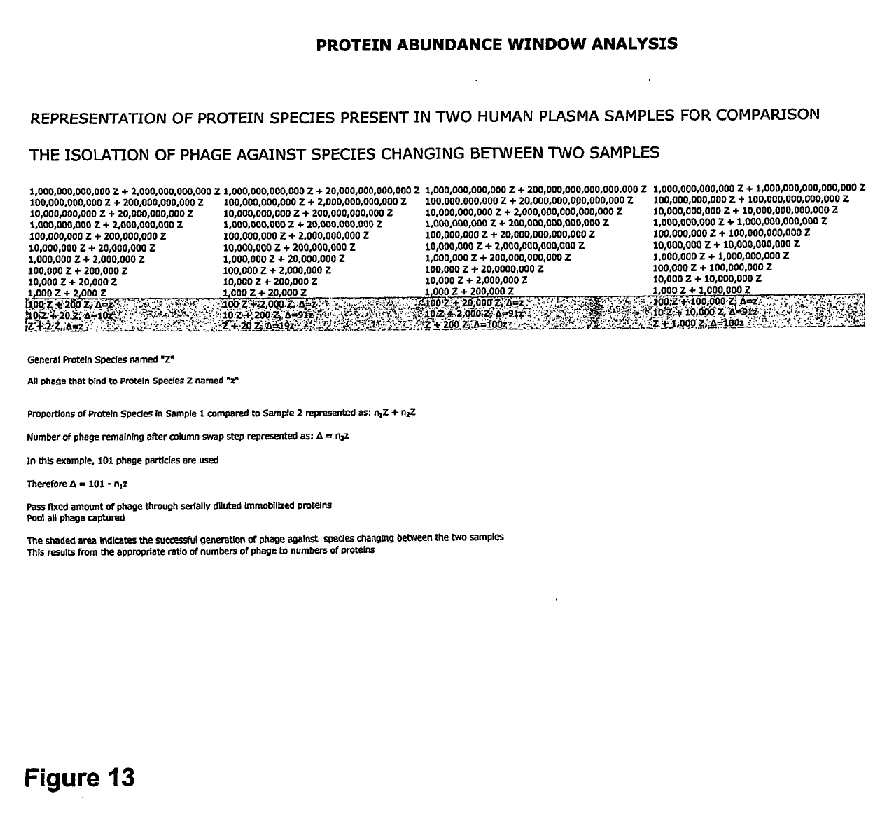

By way of illustration, FIG. 13 is a general representation of the protein species present in two human plasma samples, as an example. Please see the figure for a summary of the assumptions and representations. The figure shows the species potentially present over 12 orders of magnitude of concentration of two undiluted human plasma samples for differential analysis. Ranges in the increase of the generalized protein species Z are shown, for example, from a 2-fold to a 1,000-fold change. The figure shows what happens if the samples are subjected to the DPCP process using a phage library containing 101 copies of each phage species against each single protein species. For simplicity, it is assumed that a single phage particle will bind with 100% efficiency to its corresponding protein. Strikingly, this single concentration of phage yields specific binding species from a single Z to 100 Z, with a constant yield of phage numbers from a 200-fold excess to a 1,000-fold excess and beyond. The more abundant species above these levels of proteins are invisible to this number of phage particles. However, the next most abundant level of proteins at 1,000 Z will become amenable to analysis if the sample is diluted 1 in 10. Therefore, by preparing a series of serial dilutions of the samples, and by challenging each dilution with a new phage sample at a fixed number of particles, the whole plasma sample can be analyzed in a series of “slices” of concentration via a Protein Abundance Window. The phage isolated after the DPCP process for each slice may be pooled for the next step in the process.

Alternatively, instead of challenging a series of serial dilutions with a fixed concentration of phage, the phage-to-protein ratio can be adjusted by exposing a sample to, for example, a series of increasing concentrations of phage. At lower phage concentrations, differences in the low abundance proteins are detected. As the phage concentration increases, differences in the more abundant proteins become detectable as the number of phage exceed the numbers of the more abundant proteins.

Although Protein Window Analysis is described herein using the example of the Differential Capture process with a phage display library, one skilled in the art will appreciate that these techniques would be applicable to other types of affinity capture techniques using other types of libraries, such as those described in the following section.

Differential Small Molecule Capture Proteomics

The Differential Phage Capture Proteomics methodology as outlined above has focused on display libraries in which an amplification step links the capture of an affinity phage particle to the large-scale preparation and identification of the affinity ligand. However, other display libraries employing a variety of powerful molecular probes are amenable to the general process of Differential Capture Proteomics (DCP). Such libraries include, but are not limited to, oligonucleotide libraries (Aptamers: Selected Oligonucleotides for Therapy, J. J. Toulme, Curr. Opin. Mol. Ther. Vol 2, p 318-324 (2000); Synthetic Oligonucleotide Combinatorial Libraries and Their Applications, W. T. Markiewicz et al., Farmaco, Vol 55, p 174-177 (2000)), carbohydrate libraries (Combinatorial Synthesis of Carbohydrates, F. Schweizer, O. Hindsgaul, Curr. Opin. Chem. Biol. Vol 3, p 291-298 (1999)), and organic small molecule libraries (Structure-Based Combinatorial Library Design: Methodologies and Applications, M. P. Beavers, X. Chen, J. Mol. Graph. Model. Vol 20 p 463-468 (2002)). When such libraries are employed, there is generally no direct amplification of the captured molecules. Species captured during the Differential Capture Process are identified, for example, by mass spectrometry (Analysis and Screening of Combinatorial Libraries using Mass Spectrometry, Y. G. Shin, R. B. van Breemen, Biopharm. Drug Dispos. Vol 22, p 353-372 (2001)) and/or nuclear magnetic resonance spectroscopy (NMR Tools for Biotechnology, A. Kiefer, Curr. Opin. Biotechnol. Vol 10 p 34-41 (1999)), and then synthesized using the same synthetic methodology used to generate the original library. These additional libraries greatly extend the range of useful molecules against the difference proteins between biological samples that can be isolated, identified, and exploited.

The DPCP method is illustrated by the following example which describes an application of this technique to a pair of samples of human cerebrospinal fluid (CSF). The example is in no way intended to be limiting of the invention.

### EXAMPLE

Step I. Proteins Capture Phage Against Proteins Differing Between Samples

(a) Preparation of Protein Affinity Matrices for Two Samples (FIG. 1).

Sample Collection. Samples of human CSF are collected from two different individuals by lumbar puncture in a sterile container. The samples should immediately be placed in an ice bath and brought to the laboratory for analysis. On arrival in the laboratory, the CSF samples should be centrifuged to remove circulating cells at 2,000 g for 10 minutes at 5° C. The samples can be either processed immediately or stored at −7° C. until analysis (Sanchez, J. C. and Hochstrasser, D. F., “Proteome Analysis Protocols” in Methods in Molecular Biology, Vol. 112, Humana Press, Totowa, N.J. (1999)).

Sample Preparation. To dissociate bound proteins and other species, and to minimize protein interactions, aliquots of the samples (100 μl) are made up to a final concentration of 3M guanidine HCl and 0.2M formic acid, and incubated in ice for 60 minutes. They are then buffer exchanged using a HiTrap Desalting Column (Amersham Pharmacia Biotech) on an HPLC system at a flow rate of 5 ml/min with phosphate-buffered-saline (PBS) and collected in a final volume of 200 μl. The low molecular weight flow-through can be passed through a SepPak column and washed with PBS to isolate peptides for additional analysis if desired. If the in vivo associations of proteins are to be preserved, then the additions of guanidine HCl and formic acid are omitted, and the CSF aliquots are diluted 1:2 with PBS before the next step.

Protein Biotinylation. In the next step, the proteins in the buffer exchanged samples are biotinylated. Immediately prior to use, make a 0.5 mg/ml solution of Sulfo-NHS-LC-Biotin in 2 mM sodium acetate, pH 6.0. Add 50 μl of this solution to the desalted CSF sample and incubate in ice for 2 hours. Add 500 μl 1M Tris-HCl, pH 7.4, and incubate for a further 30 minutes to inactivate the remaining biotin linker. To remove unreacted biotin, desalt using a HiTrap Desalting Column (Amersham Pharmacia Biotech) on an HPLC system at a flow rate of 5 ml/min with TBS (50 mM Tris-HCl, pH 7.5, 150 mM NaCl) and collect in a final volume of 200 μl (Barbas, F., et al., Phage Display: A Laboratory Manual, Cold Spring Harbor, N.Y. (2001)).

Capture Column Preparation. Pass a biotinylated and desalted solution from a human CSF sample, named Sample 1, over a HiTrap Streptavidin Sepharose Column (Amersham Pharmacia Biotech) on an HPLC system equilibrated with TBS at 0.1 ml/minute. Wash this column (“Column 1”), with 20 ml of TBS at 1.0 ml/minute. Repeat this process for a second human CSF sample, named Sample 2, to yield a second column (“Column 2”).

(b) Capture Step One (FIG. 2)

Proteins Capture Phage. Pass 10³ to 10⁴ equivalents of a Phage Peptide Library (e.g., 2×10¹¹ particles for a library of 2×10⁸ clones would be 10³ equivalents) in 5 ml of TBS over each of Columns 1 and 2 on an HPLC system, at a flow rate of 40 μl/minute. Wash the columns with 20 ml TBS at a flow rate of 0.5 ml/minute.

Captured Phage are Eluted. Elute the bound phage with 1 ml Panning Elution Buffer (0.1 M HCl adjusted to pH 2.2 with glycine) at a flow rate of 0.5 ml/minute and collect fractions. Pool the fractions containing phage, buffer exchange using a HiTrap Desalting Column (Amersham Pharmacia Biotech) on an HPLC system at a flow rate of 5 ml/min with TBS, and collect in a final volume of about 1 ml. (Barbas, F. et al., Phage Display: A Laboratory Manual, Cold Spring Harbor, N.Y. (2001)).

(c) Capture Step Two (FIG. 3)

Proteins Capture Phage from Swapped Columns. Pass the eluted and buffer exchanged phage from Column 1 over Column 2 on an HPLC system, at a flow rate of 40 μl/miute. Wash the column with 20 ml TBS at a flow rate of 0.5 ml/minute. Repeat this process with the eluted and buffer exchanged phage from Column 2 over Column 1.

Captured Phage from Swapped Columns are Eluted. Elute the bound phage from Column 1 with 1 ml Panning Elution Buffer (0.1 M HCl adjusted to pH 2.2 with glycine) at a flow rate of 0.5 ml/minute and collect fractions. Pool the fractions containing phage, and neutralize with about 140 μl 1M Tris Base, pH 9.1. Verify the pH has been raised to yield a solution of pH 7.0-8.2. Repeat this process with the bound phage from Column 2.

Step II. Specific Phage Capture Difference Proteins

(a) Preparation of Phage Affinity Matrix (FIG. 4)

Pool Phage. Pool the flow-through phage from Columns 1 and 2.

Amplif Phage. Cells for Phage Infection are prepared according to the following protocol:

1. Transfer a single colony of K91 cells to a culture tube containing 2 ml of NZY. Incubate overnight at 37° C. with vigorous shaking (250 rpm).

2. Inoculate a 125-ml flask containing 20 ml of NZY with 400 μl of the overnight culture. Shake vigorously (250 rpm) at 37° C. until the cells have reached mid-log phase (OD₅₉₅=−0.45; this takes about 1.5-2 hours).

3. Use slow shaking to 100 rpm for 10 minutes to allow the bacteria to regenerate sheared F. pili. Measure the OD₅₉₅; it preferably should not be over 0.65 (best at 0.55-0.65).

4. Transfer the culture to an Oak Ridge tube and centrifuge at 600 g (2,200 rpm) in a SORVALL SS34 rotor (or equivalent) for 10 minutes at room temperature or 4° C.

5. Discard the supernatant and gently resuspend the cells in 20 ml of 80 mM NaCl.

6. Transfer the mixture to a 125-ml culture flask and shake gently (100 rpm) at 37° C. for 45 minutes.

7. Transfer the mixture to an Oak Ridge tube and centrifuge at 850 g (2,800 rpm) in an SS34 rotor (or equivalent) for 10 minutes at 4° C.

8. Pour off the supernatant and resuspend the cells gently in 1 ml of 4° C NAP buffer.

9. Keep the cells on ice when in use, and store on ice in the refrigerator. The cells are best used immediately, but will stay competent for phage infection for a few days. The cells are no longer competent if they remain aggregated after gently shaking of the tube in which they are stored. The final concentration of cells should be approximately 5×10⁹ cells/ml. (Barbas, F. et al., Phage Displa.y: A Laboratory Manual, Cold Spring Harbor, N.Y. (2001)).

Infect the Starved Cells With Eluted Phage.

1. Determine the concentration of the phage by agarose gel electrophoresis or spectrophotometry.

2. Infect cells by distributing 10 μl of diluted virions (less than or equal to 10⁶ particles) into microcentrifuge tubes or into wells across a row of a flexible ELISA plate. Add 10 μl of starved or fresh, high-density cells (˜5×10⁷ cells). Incubate at room temperature for 10-15 minutes.

3. Induce the tetracycline-resistance genes by adding 150 μl (for microtiter plates) to 1 ml (for microcentrifuge tubes) of NZY containing 0.2 μg/ml tetracycline and mixing. Incubate at 39° C. with intermittent shaking (or tapping the ELISA plate) for 30 minutes. If using 1.5 ml microcentrifuge tubes, place the tubes upside-down in a beaker and shake in an incubator. Briefly microfage the tubes before opening, and mix the cells by pipetting.

4. Dilute the infected cells in flexible microtiter plates with NZY containing 15 μg/ml tetracycline. Mix 20 μl of cells with 180 μl of NZY+tet for 1:10 dilutions, and 2 μl in 198 μl of NZY+tet for 1:1000 dilutions.

5. The concentration of tetracycline-resistant transducing units (TLU) is determined by titering. Titering of an unknown sample is usually done alongside cells infected with a positive control phage whose number of particles/ml is known and that has been previously titered. A good positive control is a stock of CsCl-purified fd-tet or f88-4 phage. Cells that have been treated with phage-dilution buffer alone, in parallel with the diluted phage samples, serve as the negative control. When counting colonies, 1 colony=1 TU.

6. For plate titering, spread 100 μl of diluted, infected cells onto NZY agar plates containing 40 μg/ml tetracycline. Spread one plate per dilution. Invert the plates and incubate overnight at 37° C.

7. For spot titering, use plates that have been dried by incubating them with their lids askew in a 37° C. incubator or sterile hood for a few hours. Before spotting, mark the plates where each spot (up to 16 per plate) will go. Carefully dot 15-20 μl of diluted phage over each spot (a multichannel pipettor can be used for this). Let the agar absorb the drops (the spots should become flat), then invert the plates and incubate overnight at 30° C.

8. For colony counting (especially from spot titers), the tetracycline-resistant colonies should be small, but visible. To prevent the overgrowth of colonies, (1) take the plates out of the 37° C. incubator before leaving for the night, let them sit overnight at room temperature, and put them back at 37° C. the following day until the colonies reach the right size; or (2) if the infections are done late at night, incubate overnight at 30° C. and have someone check the colonies at the beginning of the following day. Once the colonies have reached optimal counting size, they should be stored at 4° C. until they are counted. They can be stored for several weeks if the plates are sealed with Parafilm. (Barbas, F. et al., Phage Display: A Laboratory Manual, Cold Spring Harbor, N.Y. (2001)).

Large-Scale Preparation of Phage

1. Pick all single transfected colonies and mix each in a 1.5 ml microcentrifuge tube containing 220 μl of NZY medium. Pool contents of all tubes, and inoculate each of two 2-liter flasks containing 500 ml of NZY and 15 μg/ml of tetracycline with the diluted, pooled cells. Shake vigorously (˜200 rpm) for about 20 hours at 37° C.

2. Transfer 100 μl of culture to a 0.5 ml microcentrifuge tube, and pellet the cells in a microfuge by spinning full-speed (14,000 rpm) for 2 minutes. Transfer 20 μl of the phage-containing supernatant to a fresh 0.5 ml microcentrifuge tube containing 5 μl of 5× Lysis Mix (warm the Lysis Mix to make sure the SDS is in solution); mix by pipetting. Incubate tube in a 70° C. water bath for 15-20 minutes and microfuge briefly. Load a 15 μl sample onto a 0.8% agarose gel in 4× GBB, and run. Use a known quantity of f88.4 phage as a control (2×10¹⁰ phage particles=100 ng DNA). Run this gel either before going on in the procedure (to be sure the phage yield is good; ˜10¹² particles/ml of culture supernatant), or at the end of the procedure (to determine the percent yield of PEG-purified phage).

3. Divide the culture from Step 1 among four 250-ml centrifuge bottles (˜230 ml/bottle) Centrifuge at 2,400 g for 10 minutes at 4° C. Without disturbing the cell pellet, transfer the phage-containing supernatant into clean bottles, and recentrifuge at 6,200 g for 10 minutes at 4° C. Carefully pour the supernatant into fresh, tared, 250 ml centrifuge bottles and determine the culture volume in each bottle (1 g=1 ml).

4. Add 0.15 volume of PEG/NaCl solution to each bottle of measure supernatant. Screw caps on tightly, and mix thoroughly by inverting the bottles gently approximately 100 times. Incubate the mixtures for at least 4 hours on ice or overnight at 4° C.

5. Pellet the phage by centrifuging at 6,200 g for 40 minutes at 4° C. Discard the supernatant, being careful not to disturb the pellet. Remove the residual supernatant by briefly recentrifuging the bottles, tilting each bottle so that the pellet is opposite the remaining supernatant, and aspirating with a 1-ml pipettor.

6. Add 7.5 ml of TBS to each bottle and shake at 150 rpm in a 37° C. incubator for approximately 30 minutes to resuspend the pellets. Centrifuge briefly to drive the solution to the bottom of each bottle. Transfer the solution from all four bottles to two tared Oak Ridge tubes. Rinse each bottle with another 7.5 ml of TBS and add to the Oak Ridge tubes. Each tube should now have a total volume of 30 ml. Balance the tubes with TBS and mix the phage thoroughly by inversion.

7. Centrifuge the tubes at 10,100-22,700 g for 10 minutes at 4° C. to clear the supernatants. Transfer the supernatants to fresh, tared Oak Ridge tubes, and determine the volumes (1 g=1 ml).

8. Add 0.15 volume of PEG/NaCl solution to each tube, and invert gently approximately 100 times. Allow the phage to precipitate by incubating the tubes for at least 1 hour on ice. A heavy precipitate should appear.

9. Collect the precipitated phage by centrifuging at 10,100 g for 40 minutes at 4° C. Remove the supernatant as in Step 5 above.

10. Add 10 ml of TBS to each tube. Resuspend the phage pellet by gently vortexing, and then allowing the pellet to soften at room temperature for about 1 hour. Vortex again, and briefly centrifuge to drive the solution down. (If the phage are to be further purified on a CsCl density-gradient, add only 5 ml of TBS to each tube, resuspend the phage, and combine the two supernatants into a single Oak Ridge tube.)

11. Clear the supernatants by centrifuging the tubes at 10,100-22,700 g for 10 minutes at room temperature or 4° C. Pour the cleared supernatant from each tube into a 15-ml polypropylene snap-cap tube and store at 4° C. in the dark.

12. To determine the concentration and yield of phage particles, treat an aliquot of phage with 5× Lysis Mix (as in Step 2). Run 1- 5- and 10-μl samples on a 1.2% agarose gel in 4× GBB, using as a standard a known amount of phage treated in the same way. Include on the gel a sample from the original culture supernatant (Step 2) to calculate the percent yield. Electrophoretic analysis is also important for demonstrating that only one DNA species is present; this is especially important for fd-tet derivatives, which can delete the tetracycline genes, generating phage with approximately 6-kb genomes. The concentration of phage particles can be more accurately assessed by spectrophotometric analysis; however, this is better done with CsCl-purified phage. The infectious properties of the phage (TU/ml) can by analyzed by titering; the infectivity of fd-tet-derivatives is about 20 particles/TU, whereas that of wild-type derivatives is about 1 particle/pfu.

13. The final concentration of phage (if they are not to be CsCl-purified) should not exceed approximately 3×10¹³/ml, so once the phage concentration is known, it should be adjusted accordingly with TBS. To impede cell growth, the solution can be adjusted to a final concentration of 0.02% (w/v) sodium azide or 20 mM Na₂EDTA. The phage can be stored long term in 50% (v/v) sterile glycerol at −18° C. (Barbas, F. et al., Phage Display: A Laboratory Manual, Cold Spring Harbor, N.Y. (2001)).

Phage Purification on CsCl Gradients

1. Following the large-scale preparation of phage above, the final volume of each preparation should be 10 ml.

2. Weigh out 4.83 g of CsCl into a tared 50-ml beaker. Tare the beaker again, and add the resuspended phage. Add TBS to a final weight of 10.75 g (over and above the weight of the vessel and CsCl: phage+TBS=10.75 g). This should give 12 ml of a 31% (w/w) solution of CsCl with a density of 1.3 g/ml. The density of the solution can be checked by weighing 1 ml in a tared beaker or plastic cup, and then returning it to the beaker it came from (be sure to first check that the pipet on that setting weighs 1 ml of water at 1 g). If necessary, adjust the density to 1.3 g/ml with CsCl or buffer.

3. Transfer the volume of each beaker into a polyallomer tube. Be sure that the tubes are filled to the top (as they can collapse during centrifugation), and if necessary, add extra volume with a 31% (w/w) solution of CsCl in TBS. Balance the tubes, by transferring solution from one to another, or by adding more CsCl solution to one tube. Place the tubes in a SW40 swinging-bucket rotor and centrifuge at 37,000 rpm for 48 hours at 5° C. For the 70.0 Ti rotor, spin at 58,000 rpm for 20 hours.

4. Carefully remove the tubes from the rotor, place in a rack, and set them up, one at a time, in a clamp stand. Illuminate the clamped tube from the top with a strong, visible light source (such as a halogen desk lamp). There will be two bands toward the top of the tube. The phage band, which will be faint, bluish, and homogeneous (smoky looking), should be just visible above a narrow, stringy, flocculent, opaque white band (which is probably PEG). In a good phage preparation, the phage band is about 5 mm in width, and its density is approximately 1.33 g/ml.

5. Attach a sterile pipet tip to an aspirator pump and adjust the aspirator to a moderate speed. Hold the pipet tip at the meniscus, and aspirate the fluid overlying the phage band to within 2 mm of the upper edge, being careful not to disturb the phage band. Withdraw the phage band with a polyethylene transfer pipet (preferably sterile), or, preferably, a sterile, glass transfer pipet attached to a peristaltic pump. The phage band will be viscous; try to avoid the flocculent band that lies underneath. If using a quickseal polyallomer tube, remove the band with a syringe, as for CsCl-purified plasmid.

6. Transfer the extracted phage bands to a 26-ml, screw-cap polycarbonate centrifuge tube for the Beckman 60 Ti rotor. 4-6 phage bands can be pooled in a single bottle. Fill the tube to the shoulder with TBS, close the cap firmly, and invert repeatedly to mix. For centrifugation, balance against another tube filled with water.

7. Centrifuge the tubes in a Beckman 60 Ti fixed-angle rotor at 50,000 rpm for 4 hours at 4° C. to pellet the phage. Pour off and discard the supernatant, recentrifuge the pellet briefly at a low speed on a tabletop centrifuge, and discard the remaining supernatant, with the pellet pointed away from the liquid.

8. Resuspend the pellet in 10 ml of TBS; vortex gently, centrifuge briefly in a table-top centrifuge to drive the solution down, and allow the pellet to soften overnight at 4° C. Vortex to dissolve the pellet.

9. Top the bottle with TBS, recentrifuge to pellet the phage, and remove the supernatant as in Step 7. (Note: This step is optional, giving somewhat purer phage.)

10. Resuspend the pellet in TBS as in Step 9, using 12 ml of TBS per liter-equivalent of starting culture; this gives an anticipated concentration of 3×10¹³ virions/ml. Transfer the phage to a sterile Oak Ridge tube and centrifuge at 6,500 g for 10 minutes.

11. Transfer the phage-containing supernatant to a 15-ml polypropylene, snap-cap tube. At this point, sodium azide can be added as a preservative to the cooled phage to a final concentration of 0.02% (w/v). Alternatively, Na₂EDTA can be used at a final concentration of 20 mM.

12. Measure the concentration of phage particles spectrophotometrically, and/or by agarose gel electrophoresis.

13. Dilute the phage 10⁻⁷, 10⁻⁸ and 10⁻⁹ in TBS/gelatin and titer. Include the proper positive and negative controls. A good infective titer (TU/ml) is approximately 5% of the concentration of physical particles (virions/ml).

14. Store the phage at 4° C. away from light, or in 50% glycerol at −18° C. for long-term storage. Under these conditions, titers are stable for at least several years. (Barbas, F. et al., Phage Display: A Laboratory Manual, Cold Spring Harbor, N.Y. (2001)).

(b) Preparation of Identical Phage Columns

Cross-Linking. Mix one volume of phage (all subsequent volumes in the paragraph are relative to this volume) at a concentration corresponding to 1.26 mM pVIII subunits in water with 0.15 volume 0.5 M NaCl and 0.15 volume 1 M NaH₂PO₄ (pH adjusted to 6.9 with NaOH). To this solution add 0.0026 volume of 76.3 mg/ml NHS-dextran. Immediately mix the reaction mixture by vortexing, add 0.15 volume 50% PEG, and vortex again. The phage will precipitate in the PEG solution (final concentration 5%). Rotate the reaction mixture for 12-16 hours in a sealed tube at room temperature, and quench unreacted N-hydroxy-succinamide by adding 8 volumes 1M ethanolamine (pH adjusted to 9 with HCl) and 0.89 volumes 5 M NaCl. Continue rotation for an additional 1-2 hours at room temperature. Dilute the cross-linked phage in TBS (at least 10 volumes) and wash 5-6 times by centritugation, aspiration or decanting the supernatant, and resuspend the pellet in fresh TBS. Suspend the final pellet in 10 volumes TBS and store at 4° C.

Column Preparation. Pack two 0.5 ml columns (Amersham Pharmacia Biotech, HR 5/2, Code 18-0382-01), each with cross-linked phage equivalent to about 2×10¹³particles. Equilibrate the columns on an HPLC system with TBS (Other columns or non-column batch methods are also appropriate).

Sample Preparation. To dissociate bound proteins and other species, and to minimize protein interactions, make up aliquots (1.5 ml) of the original human CSF Samples 1 and 2 to a final concentration of 3M guanidine HCl and 0.2M formic acid, and incubate in ice for 60 minutes. Then buffer exchange them using a HiTrap Desalting Column (Amersham Pharmacia Biotech) on an HPLC system at a flow rate of 5 ml/min with phosphate-buffered-saline (PBS) and collect in a final volume of 2 ml. Pass the low molecular weight-flow-through over a SepPak column and wash with PBS to isolate peptides for additional analysis if desired. If the associations of proteins in the original sample are to be preserved, then omit the additions of guanidine HCl and formic acid, and dilute the CSF aliquots 1:2 with PBS before the next step.

(c) Capture Step Three (FIG. 5)

Sample Difference Proteins Captured By Phage Columns. Pass 1.5 ml of Samples 1 and 2 over a dedicated cross-linked phage column in TBS on an HPLC system at a flow rate of 20 μl/minute. Keep the flow-throughs, which are the depleted protein samples and which will be used in subsequent cycles. Wash the columns with 20 ml TBS at a flow rate of 0.5 ml/minute.

Step III: Quantification and Identification of Difference Proteins (FIG. 6)

Isolate Difference Proteins Using Phage Affinity Columns. Elute the bound proteins from the columns with 1 ml Panning Elution Buffer (0.1 M HCl adjusted to pH 2.2 with glycine) at a flow rate of 0.5 ml/minute and collect 100 μl fractions. Pool the fractions and neutralize with about 140 μl 1M Tris Base, pH 9.1. Verify the pH has been raised to yield a solution of pH 7.0-8.2.

(a) Analysis by Cation Exchange/Reversed-Phase Columns and Electrospray Mass Spectrometry

Digestion of Proteins to Peptides. Lyophilize the samples prepared above, and resolubilize in 8 M urea, 200 mM NH₄HCO₃, and 20 mM CaCl₂ and quantify using a Bradford assay. Add Endoprotease Lys-C (Boehringer-Mannheim) to a final substrate-to-enzyme ratio of 100:1, and incubate at 37° C. for 15 hours. Dilute the Lys-C digestion fourfold with water, and add modified trypsin (Boehringer-Mannheim) to a final substrate-to-enzyme ratio of 50:1. Incubate the trypsin digestion mixture at 37° C. for 15 hours. Desalt the peptide mixture on a reversed-phase column, lyophilize, and resuspend in 5 mM K₂HPO₄, 5% acetonitrile (pH 3) (other chemical and enzymatic digestion methods are also appropriate).

(b) 2D Chromatographic Separation of Peptides.

Biphasic Microcapillary Column. Construct the biphasic column, by pulling a fused-silica capillary (100 μm i.d.×365 μm o.d.) with a CO₂-based laser puller to make a fritless column. Pack the column first with 8 cm of 5 μm C18 RP particles (218TP C18 Vydac) and then with 4 cm of 5 μm strong cation exchange particles (PolySULFOETHYL Aspartamide; Poly LC).

Peptide Separation. Load the peptide mixtures onto the biphasic microcapillary column. Displace peptide fractions from the SCX to the RP particles using the following salt step gradients: (1) 0% (2) 0-10% (3) 10-20% (4) 20-30% (5) 30-40% (6) 40-100% of SCX-B′, and (7) 100% SCX-C′. Elute peptides from the RP particles into the mass spectrometer using a linear gradient of 0-60% RP-B over 30 minutes at 300 nl/minute. Mobile-phase buffers are, for RP-A buffer, 0.5% acetic acid, 5% acetonitrile; for RP-B, 0.5% acetic acid, 80% acetonitrile; for SCX-B′, 0.5% acetic acid, 5% acetonitrile, 250 mM KCl; for SCX-C′, 0.5% acetic acid, 5% acetonitrile, 500 mM KCl (other separation methods are also appropriate).

Mass Spectrometric Analysis. Perform mass spectrometric analysis on a Finnigan LCQ ion trap mass spectrometer (Finnigan Corp., San Jose, Calif.) run and operate as described. Directly couple an Integral chromatography workstation (PE Biosystems, Foster City, Calif.) to an LCQ ion trap mass spectrometer equipped with an electrospray ion source. Operate the electrospray needle with a voltage differential of 5.5 kV, and hold the heated desolvation capillary at 250° C.

Identify and Quantify Via Mass Fingerprinting and Sequencing With Electrospray Mass Spectrometry. For automated spectrum and data analysis, process each raw tandem spectrum as described here. First identify spectra derived from single or multiply charged parent ions. Correlate processed tandem mass spectra with standard ORFs using the program SEQUEST running on a DEC Alpha workstation. Perform all searches without considering the protease used because many proteins in the mixtures do not digest to completion. For multiply charged peptides, use the following criteria to determine whether to select the +2 or +3 charge state: (1) Choose a particular charge state if the cross-correlation score is greater than or equal to 1 U more than that of the other charge state. (2) Assign a score to each charge state (+5 for tryptic start, +5 for tryptic end, +2 if the cross-correlation score is greater than the other charge state, +2 if the preliminary score ranking is less than 50, and the charge state with the highest score is chosen. Use the selected charge of the peptide in the final protein identification analysis and the SEQUEST output from the other charge state discarded. Filter the correlation results using the value of the cross-correlation score and the matched sequence for each spectrum. For singly charged peptides, retain spectra with a cross-correlation score to a tryptic peptide great than or equal to 1.5. For multiply charged peptides, retain spectra with a cross-correlation to a tryptic peptide greater than or equal to 2. Eliminate all spectra with cross-correlation scores not meeting these criteria from further consideration. For the protein identifications, sort the filtered results to show unique peptide sequences that are derived from the same annotated ORFs in the genome. Protein identifications based on mass spectra correlating to one or more unique tryptic peptides are considered valid identifications. Single peptides that alone identify a protein are manually validated after meeting the following criteria. First, the SEQUEST cross-correlation score must be greater than 1.5 for a +1 tryptic peptide or greater than 2 for a +2 or +3 tryptic peptide. Second, the MS/MS spectrum must be of good quality with fragment ions clearly above baseline noise. Third, there must be some continuity to the b or y ion series. Fourth, the y ions that correspond to a proline residue should be intense ions. Fifth, unidentified, intense fragment ions either correspond to +2 fragment ions or the loss of one or two amino acids from one of the ends of the peptide. After going through this process, the confidence level of protein identification is high. For proteins common to Samples 1 and 2, approximate relative abundance ratios can be determined using Finnigan LCQUAN software and peak heights of molecular ions (Link, A. J., et al., Direct Analysis of Protein Complexes Using Mass Spectrometry. Nature Biotechnology 17:676-682 (1999)).

Step IV: Isolation of Affinity Reagents Against Difference Proteins

Pool the flow-through phage as indicated in FIG. 7. Amplify phage, prepare phage affinity matrices and phage columns, and capture difference proteins as described in steps II(a)-(c) above. The number of proteins identified in step III (i.e., the step involving the quantification and identification of difference proteins) (FIG. 6) is a guide to the numbers of distinct phage clones that need to be tested.

Assignment of phage clone specificity to identified protein is carried out as follows:

1. Isolate Captured Protein Using Phage Affinity Columns. Elute the bound protein from the column with 1 ml Panning Elution Buffer (0.1 M HCl adjusted to pH 2.2 with glycine) at a flow rate of 0.5 ml/minute and collect 100 μl fractions. Pool the fractions and neutralize with about 140 μl 1M Tris Base, pH 9.1. Verify the pH has been raised to yield a solution of pH 7.0-8.2.

2. Identify Eluted Protein. The eluted protein is identified using an LCQ Mass Spectrometer as described in step m above, and illustrated in FIG. 6, except that since the major component should be a single protein, the sample can be directly sprayed into the instrument.

3. Assign Phage Clone to Identified Protein. The association of a particular phage clone with the identity of the protein it captures, allows its specificity to be defined. The phage provide a specific affinity reagent against the protein, and if necessary, the identity of the peptide or antibody can be determined to allow the production of high purity peptide or antibody, via well established methods. Multiple distinct phage clones may be isolated which bind to the same protein. Their affinities may determine their utility.

Step V: Depletion of Most Abundant Common Proteins (FIG. 8)

Prepare protein affinity matrices and perform initial capture step as described in steps I(a) and (b) above. Elute the bound phage as indicated in FIG. 2 with 1 ml Panning Elution Buffer (0.1 M HCl adjusted to pH 2.2 with glycine) at a flow rate of 0.5 ml/minute and collect fractions. Pool the fractions containing phage, buffer exchange using a HiTrap Desalting Column (Amersham Pharmacia Biotech) on an HPLC system at a flow rate of 5 ml/min with TBS, and collect in a final volume of about 1 ml.

Most Abundant Common Proteins Captured By Phage Columns. Pool the eluted phage from Columns 1 and 2. Amplify phage, prepare phage affinity matrices and phage columns, and capture difference proteins using the procedures described in steps II(a)-(c) above. Pass 1.5 ml of Samples 1 and 2 over a dedicated cross-linked phage column in TBS on an HPLC system at a flow rate of 20 μl/minute. Keep the flow-throughs, which are the depleted protein samples and which will be used for the next cycle (FIG. 9). Wash the columns with 20 ml TBS at a flow rate of 0.5 ml/minute.

Isolate Difference Proteins Using Phage Affinity Columns. Elute the bound proteins from the columns with 1 ml Panning Elution Buffer (0.1 M HCl adjusted to pH 2.2 with glycine) at a flow rate of 0.5 ml/minute and collect 100 μl fractions. Pool the fractions and neutralize with about 140 μl 1M Tris Base, pH 9.1. Verify the pH has been raised to yield a solution of pH 7.0-8.2. The eluted proteins can be further purified if necessary, and can be an invaluable source of “native” material for further structural and functional studies, including providing a standard for the development of immunoaffinity based assays using the specific affinity reagents isolated from DPCP.

Step VI. New Cycle with Less Abundant Common Proteins

Using the flow-throughs from Step V, which are the depleted protein samples, the cycle described above is repeated (FIG. 9).

Step VII. Continuing Depletion of Abundant Common Proteins

Steps V and VI can be continuously repeated until the limits of detection of the analytical device have been reached.

### Other Embodiments

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein. Such equivalents are intended to be encompassed in the scope of the present invention.

All publications and patents mentioned in this specification are herein incorporated by reference.

Other embodiments are within the claims.

What is claimed is:

1. A method for determining an abundance of a biomolecule in a biological sample, said method comprising the steps of:
(a) adhering a first biological sample to a first support to create a first matrix comprising one or more biomolecules from said first sample;
(b) adhering a second biological sample to a second support to create a second matrix comprising one or more biomolecules from said second sample;
(c) exposing a library of binding species at least one time to said first matrix to create a first product comprising one or more binding species of said library; and
(d) exposing the first product at least one time to said second matrix to create a second product, wherein a binding species present or absent in said second product is indicative of the abundance of said biomolecule in said first biological sample relative to said second biological sample.

2. The method of claim 1, further comprising the steps of:
(e) exposing said library to said second matrix to create a third product comprising one or more binding species of said library; and
(f) exposing the third product at least one time to said first matrix to create a fourth product, wherein a binding species present or absent in said fourth product is indicative of the abundance of said biomolecule in said second biological sample relative to said first biological sample.

3. The method of claim 2, further comprising the step of
(g) comparing said second and fourth products to determine the abundance of said biomolecule in said first sample relative to said second sample.

4. The method of claim 2, further comprising the steps of:
(g) combining the second and fourth products to produce a pooled product;
(h) adhering at least a portion of the pooled product to a third support to provide a third matrix;
(i) exposing said first biological sample at least one time to said third matrix to provide a fifth product;
(j) exposing said second biological sample at least one time to said third matrix to provide a sixth product; and
(k) comparing said fifth and sixth products to determine the abundance of said biomolecule in said first sample relative to said second sample.

5. The method of claim 1, wherein said first product consists essentially of one or more binding species that bind to said first matrix, or said first product consists essentially of one ore more binding species that do not bind to said first matrix.

6. The method of claim 5, further comprising controlling the relative amount of binding species exposed to said one or more biomolecules of said first matrix in step (c).

7. The method of claim 6, wherein said controlling occurs by preparing a dilution of said first biological sample and creating said first matrix from said dilution.

8. The method of claim 7, wherein steps (a)-(k) are repeated, wherein in each iteration, a different dilution is employed to create said first matrix.

9. The method of claim 6, wherein said controlling occurs by preparing a dilution of said library and exposing said dilution to said first matrix.

10. The method of claim 9, wherein steps (c)-(k) are repeated, wherein in each iteration, a different dilution is exposed to said first matrix.

11. The method of claim 1, wherein said second product consists essentially of one or more binding species that bind to said second matrix, or said second product consists essentially of one or more binding species that do not bind to said second matrix.

12. The method of claim 11, further comprising controlling the relative amount of binding species exposed to said one or more biomolecules of said second matrix in step (e).

13. The method of claim 12, wherein said controlling occurs by preparing a dilution of said second biological sample and creating said second matrix from said dilution.

14. The method of claim 13, wherein steps (a)-(k) are repeated, wherein in each iteration, a different dilution is employed to create said second matrix.

15. The method of claim 12, wherein said controlling occurs by preparing a dilution of said library and exposing said dilution to said second matrix.

16. The method of claim 15, wherein steps (c)-(k) are repeated, wherein in each iteration, a different dilution is exposed to said second matrix.

17. The method of claim 2, wherein said third product consists essentially of one or more binding species that bind to said second matrix, or said third product consists essentially of one or more binding species that do not bind to said second matrix.

18. The method of claim 2, wherein said fourth product consists essentially of one or more binding species that bind to said first matrix, or said fourth product consists essentially of one or more binding species that do not bind to said first matrix.

19. The method of claim 4, wherein said fifth product consists essentially of one or more biomolecules that bind to said third matrix, and said fifth product consists essentially of one or more biomolecules that do not bind to said third matrix.

20. The method of claim 4, wherein said sixth product consists essentially of one or more biomolecules that bind to said third matrix, or said sixth product consists essentially of one or more biomolecules that do not bind to said third matrix.

21. The method of claim 1, wherein said biomolecule is a protein, nucleic acid, carbohydrate, fatty acid, lipid, steroid, prostaglandin, prostacyclin, or small organic molecule.

22. The method of claim 1, wherein, prior to step (a), one or more biomolecules in said first biological sample is derivatized with a derivatizing agent in order to enable adhesion to said first support.

23. The method of claim 22, wherein the extent of derivatization is controlled by the type of derivatizing agent, the concentration of the derivatizing agent or the one or more biomolecules, the temperature, or time of reaction.

24. The method of claim 1, wherein, prior to step (b), one or more biomolecules in said second biological sample is derivatized in order to enable adhesion to said second support.

25. The method of claim 24, wherein the extent of derivatization is controlled by the type of derivatizing agent, the concentration of the derivatizing agent or the one or more biomolecules, the temperature, or time of reaction.

26. The method of claim 1, wherein said library is a peptide-nucleic acid coupled library, a nucleic acid library, a carbohydrate library, or a small organic molecule library.

27. The method of claim 1, wherein said peptide-nucleic acid library is a phage display library.

28. The method of claim 1, wherein said phage display library is an antibody library, a recombinant display library, or a synthetic peptide library.

29. The method of claim 1, wherein said first biological sample is from a first organism, and said second biological sample is from a second organism.

30. The method of claim 1, wherein said first and second organisms are humans.

31. The method of claim 1, wherein said first organism is diseased, and said second organism is healthy.

32. The method of claim 1, wherein said first organism has been exposed to a chemical agent, and said second organism has not been exposed to said chemical agent.

33. The method of claim 1, wherein said biological sample is selected from the group consisting of a tissue, a bodily fluid, a cultured cell, and an organ.

34. The method of claim 1, wherein said tissue or said cultured cell is selected from the group consisting of epithelial, connective, muscle, and nerve tissues or cells.

35. The method of claim 1, wherein said body fluid is selected from the group consisting of cerebrospinal fluid (CSF), blood, saliva, mucous, tears, pancreatic juice, seminal fluid, sweat, milk, bile, plasma, serum, lymph, urine, pleural effusions, bronchial lavage, ascites, and synovial fluid.

36. The method of claim 1, wherein said bodily fluid is CSF.

37. The method of claim 1, wherein said organ is selected from the group consisting of skin, bone, cartilage, tendon, ligament, skeletal muscle, smooth muscle, heart, blood, blood vessel, brain, spinal cord, peripheral nerve, nose, trachea, lung, mouth, esophagus, stomach, intestine, kidney, uterus, ureters, urethra, bladder, hypothalamus, pituitary, thyroid, pancreas, adrenal gland, ovary, oviduct, vagina, mammary gland, testicle, seminal vesicle, penis, lymph, lymph node, lymph vessel, white blood cell, T-cell and B-cell.

38. The method of claim 1, wherein said first matrix comprises one or more biomolecules from said first biological sample covalently bound to said first support.

39. The method of claim 1, wherein said second matrix comprises one or more biomolecules from said second biological sample covalently bound to said second support.

40. The method of claim 4, wherein said third matrix comprises one or more binding species from said library covalently bound to said third support.

41. The method of claim 1, wherein prior to step (a), said first biological sample is treated to denature said biomolecule.

42. The method of claim 1, wherein prior to step (b), said second biological sample is treated to denature said biomolecule.

43. The method of claim 1, wherein said exposing in step (c) or step (d) occurs more than once.

44. The method of claim 2, wherein said exposing in step (e) or step (f) occurs more than once.

45. The method of claim 4, wherein said exposing in step (i) or step () occurs more than once.

46. The method of claim 1, wherein said first, second, or third support comprises a column.

47. The method of claim 1, wherein said first, second, or third support comprises a chip.

48. The method of claim 3, wherein said comparing comprises mass spectrometry.

49. The method of claim 3, wherein said comparing comprises nuclear magnetic resonance spectroscopy.

50. The method of claim 4, wherein prior to step (h) said pooled product is amplified.

51. The method of claim 1, wherein a difference in the relative abundance of said biomolecule between said first and second biological samples is indicative of a candidate therapeutic target.

52. The method of claim 1, wherein a difference in the relative abundance of said biomolecule between said first and second biological samples is indicative of a disease state.
